(19) United States
(12) Patent Application Publication
(10) Pub. No.: US 2008/0103384 A1
(43) Pub. Date: May 1, 2008

(54) Medical instrument and device for creating sectional tissue images

(75) Inventor: Marcus Pfister, Bubenreuth (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/977,112

(22) Filed: Oct. 23, 2007

(30) Foreign Application Priority Data
Oct. 27, 2006 (DE) 102006050886.6 DE

(51) Int. Cl. A61B 1/00

(52) U.S. Cl. 600/411; 600/424; 600/443; 600/478

(57) ABSTRACT

A medical instrument for introduction into the body of a living being is provided. The instrument including a first signal line and is connected for signal transmission to a signal detection device for generating sectional tissue images. Additionally, the instrument includes a second signal line for detection of fluorescence light.

### CROSS REFERENCE TO RELATED APPLICATIONS

This application claims priority of German application No. 102006050886.6 DE filed Oct. 27, 2006, which is incorporated by reference herein in its entirety.

### FIELD OF INVENTION

The invention relates to a medical instrument and a device for creating sectional tissue images.

### BACKGROUND OF INVENTION

Such a medical instrument is for example known from U.S. Pat. No. 6,377,048 B1. In this document a catheter is provided with an NMR device for generating and detecting NMR signals produced through magnetic resonance of the atomic nucleus. The NMR device enables sectional images of tissue radially surrounding the catheter to be produced. This especially enables undesired deposits in vessels to be made visible.

In addition it is also possible to produce sectional images of tissue using optical coherence tomography or ultrasound tomography. The method of combining an optical method using near infrared light with an image generation method based on ultra sound for improving the production of sectional tissue images is further known from Zhu, Q. et al.: “Imager that combines near-infrared diffusive light and ultrasound”, OPTICS LETTERS, Vol. 24, No. 15 (1999), pages 1050 to 1052.

Fluorescing metabolic markers which specifically bind to particular pathogenic area of the tissue are known from Umar Mahmood et al.: “Near Infrared Optical Imaging of Protease Activity for Tumor Detection”, Radiology 213:3 (1999), pages 866 to 870. Such pathogenic areas of the tissue can for example involve tumors, inflammations, sections of vessels affected by deposits or plaque or other seats of illness. By exciting such markers with light of a suitable wavelength pathogenic tissue sections can be made visible and removed in an operational intervention.

The dissertation by Grigorios Valassisin which appeared in 2004 entitled, “Influence of photo dynamic therapy on neointima imaging before and after stent implantation in the arteria femoralis of the domestic pig” discloses that medicaments can be specifically bound to deposits located in veins and subsequently released through irradiation with light to destroy the deposits.

### SUMMARY OF INVENTION

The object of the present invention is to specify a medical instrument and a device for generation of sectional tissue images in which healthy and pathogenic tissue sections can be distinguished in a minimally invasive manner. According to a further aim of the invention the pathogenic tissue is also to be able to be destroyed or dissolved with the method.

This object is achieved by the claims.

According to the invention there is provision, in the elongated element, for a second signal line embodied as an optical fiber routed to its free end to be provided for transmission of fluorescence light created inside the body.—This enables healthy and pathogenic sections of tissue in which fluorophoric markers are selectively accumulated, to be distinguished for display in a minimally invasive way. By inventively combining in the proposed medical instrument a signal detection device known per se for detecting first signals for generating a tissue section with a second signal line embodied as a optical fiber for transmission of fluorescence light generated inside the body, it is possible in an especially simple and low-cost manner to correlate the image information transferred over the two signal lines and to process it to form an overall image with a high diagnostic information content.

In the sense of the present invention a “medical instrument” is especially to be understood as a catheter or an endoscope. Such cases involve a mostly flexible tubular or hose-type instrument, with which the interior of the organism of a living being can be investigated. Examinations of hollow organs and vessels as well as micro-operative interventions are especially possible with flexible catheters.

According to an advantageous embodiment of the invention a third signal line embodied as an optical fiber is provided in the elongated element routed up to its free end for transmission of light of a predetermined wavelength (I) for excitation of fluorophores accommodated inside of the body and/or (ii) for release of medicaments provided inside of the body.—Obviously it is also possible to use the second signal line proposed in the invention and embodied as an optical fiber for this purpose. In this case a corresponding device is to be provided at the end opposite to the free end of the second signal line, with which alternately fluorescent light can be detected and light with a different wavelength from the fluorescent light can be coupled into the second signal line for excitation of fluorophores or for releasing medicaments. This type of alternate coupling-in and/or detection can easily be implemented with conventional technical means, especially using filters and/or optical switches and such like. By provision of the third signal line proposed above however it is advantageously possible to dispense with such a device for alternate coupling-in/coupling-out of signals or such a device can be simplified.

According to a further embodiment of the invention there is provision for the signal detection device to be a device for detecting signals for creation of sectional tissue images by means of optical coherence tomography (OCT). Catheters for creating sectional images of tissue by means of OCT are generally known from the prior art. The signal detection device in this case includes a reflection means provided at the free end of the first signal line, e.g. a mirror.

According to a further embodiment there is provision for the signal detection device to be a device for detection of signals for creating sectional images of tissue by means of ultrasound tomography (IVUS). Catheters for producing sectional images of tissue using ultrasound tomography are known from the prior art from U.S. Pat. No. 5,345,940 for example, the disclosure content of which is herewith included in this document. They stand out form the above-mentioned OCT catheters especially by enabling sectional images of tissue to be created with a greater penetration depth. For creating sectional images of tissue by means of IVUS the signal detection device and the first signal line connected to it can be rotatable. The signal detection device in this case involves an ultrasound transceiver, for example a piezo element used alternately as transmitter and receiver. The first signal line is in this case embodied as an electrical line. The signal detection device can however also feature a plurality of radially arranged ultrasound transceivers. In this case the signal detection device is embodied as a stationary device.

According to a further embodiment the signal detection device is a device for detecting signals for generating sectional images of tissue by means of magnetic resonance tomography (IVMRI). Catheters for creating sectional images of tissue by means of IVMRI are generally known from the prior art. The reader is referred for example to the publications U.S. Pat. No. 6,377,048 and also U.S. Pat. No. 6,600,139 referred to above of which the disclosure content is herewith included here. For creating sectional images of tissue by means of IVMRI a static magnetic field can be applied outside the body. It is however also possible to create the static magnetic field in the area of the free end of the catheter. IVMRI catheters make possible an especially good display of soft tissue and enable medical information to be obtained about deposits or plaque in vessels.

Advantageously the first signal line and the signal detection device permanently connected to it are accommodated rotatably in a tube forming the elongated element, preferably a flexibly embodied tube. Such a rotatable mounting of the signal detection device has been proven especially in OCT or IVUS catheters. It has proved especially useful in this case to connect the second and if necessary third signal line permanently to the first signal line. In this case a line group formed from the signal lines can be rotated in a simple manner with the same angular speed. Signals obtained at the free end of the signal lines allow images to be produced which provide a plurality of different information relevant to diagnosis in an especially simple manner.

According to a further embodiment of the invention a positioning device is provided at the free end for determining the position in a predetermined three-dimensional coordinate system. Such a positioning device advantageously includes a number of position sensors which are expediently accommodated in the area of the free end. Catheters with the above-mentioned positioning device and method for determining the position of the free end of the catheter in a three-dimensional coordinate system are generally known from the prior art. The reader is referred to examples in DE 198 52 441 A1 as well as DE 200414892 36 743 198 52 467 A1 of which the disclosure content is included to this extent. Other positioning devices are known for example from DE 695 14 238 T2, EP 1 034 738 B1 or EP 0 993 801 A1. In this case the position sensors are embodied as magnetic or electromagnetic transmitters or receivers which interact with an external magnetic field. As a result of the interaction a position of a position sensor provided at the free end of the catheter can be deduced in a three-dimensional coordinate system. This makes it possible to follow exactly a movement of the medical instrument, e.g. in a vessel. The proposed position sensors in particular make possible an approximation of the center line of the vessel and from this an exact production of 3D images of the vessel.

According to a further embodiment a deflection means can be accommodated in the area of the free end. The deflection means can include at least one, preferably several magnets. The magnet can be a permanent magnet and/or electromagnet. In this case a magnetic field generated by at least two permanent magnets and/or electromagnets can have be aligned differently. With the proposed deflection means it is possible, on application of suitable external magnetic fields, to defect the—in this case flexibly embodied—free end of the catheter in a desired direction. This facilitates the guidance of the catheter on a predetermined path in the vascular system.

According to a further embodiment of the invention there is provision in the elongated element to provide a line coming out in the area of the free end for carrying a fluid, especially a contrast means. The proposed line makes it possible for example to convey liquids for increasing the contrast during creation of images using ultrasound tomography. Furthermore medicaments, fluorescent markers, tracers or such like can be delivered via the proposed line.

According to a further measure of the invention a device for generation of sectional images of tissue is provided, comprising:
the inventive medical instrument,
a device for creating a sectional image of tissue using the first signals detected with the signal detection and transferred via the first signal line and
a device for creating a fluorescence image using the fluorescent light transferred via the second signal line.

The proposed device makes it possible to create sectional images of tissue as well as to create almost simultaneously a fluorescence image of the same area of tissue using the inventively proposed medical instrument. In this way valuable diagnostic information can be obtained quickly and simply.

According to an especially advantageous embodiment a device is provided for overlaying the sectional tissue image and the fluorescence image. This enables the information obtained to be presented in a single image. This image involved can be a two- or also three-dimensional image. Such an image makes possible an exact diagnosis as well as an effective minimally-invasive therapy.

According to a further embodiment a light generation device connected to the third signal line for generating light of a predetermined wavelength or of a predetermined wavelength range is provided. The third signal line can however also be omitted. In this case a light generation device connected to the second signal line for pulsed creation of light of a predetermined wavelength or of a predetermined wavelength range can be provided. During the period in which the light generation device is switched off the fluorescence light transferred via the second signal line can be detected and evaluated.

The light of predetermined wavelength or of a predetermined wavelength range involves either a light suitable for excitation of fluorophores located in the body or also light with which medicaments accumulated inside the body can be released locally. A photodynamic therapy (PDT) is thus especially possible with the proposed device. For alternate use of the second signal line, i.e. the detection of fluorescence light and the coupling-in of light of a predetermined wavelength, filters and optical switches known from the prior art as well as suitable clock generators can be used.—When the proposed third signal line is used, a clocked operation of the second signal line is not absolutely necessary.

The first signal line can be a component of a conventional device for generation of a sectional tissue image according to one of the following methods: Optical Coherence Tomography, Ultrasound Tomography and Magnetic Resonance Tomography.

According to a further embodiment of the invention a position determination device is provided for determining a position of the positioning device in a three-dimensional coordinate system. This enables an exact correlation of the images obtained with further images, obtained by x-ray methods for example. This also makes it possible to produce exact three-dimensional images, for example of vessels, through which the inventive medical instrument will be moved. Fluorescing—and thereby pathogenic—areas of tissue can be indicated in such 3D images for example by showing them in the wrong color.

Expediently a device for generating a three-dimensional image on the basis of the signals delivered by a position determination device is provided for this purpose.

Furthermore a rotation device for rotating the first signal line as well as the signal detection device can be provided. Advantageously the second, and if nec. the third signal line, are able to be rotated together with the first signal line with rotation device.

Furthermore a deflection device can be provided for deflecting the deflection means in accordance with a predeterminable program. This involves a magnet known from the prior art with which the free end of the catheter is able to be deflected in a predetermined direction depending on the length of advance of the catheter.

Finally a fluid feed device able to be connected to the line for conveying fluid can be provided. This can involve a pump or similar, with which a predetermined volume, preferably at a predetermined rate, can be fed through the line into the body.

### BRIEF DESCRIPTION OF THE DRAWINGS

Exemplary embodiments of the invention are explained in greater detail below with reference to the drawing. The figures show:

FIG. 1 a schematic sectional view of a catheter,

FIG. 2 a schematic block diagram of the major components of first image generation device,

FIG. 3 a schematic block diagram of a second image generation device and

FIG. 4 a schematic diagram of an overlaid image generated with the image generation device.

### DETAILED DESCRIPTION OF INVENTION

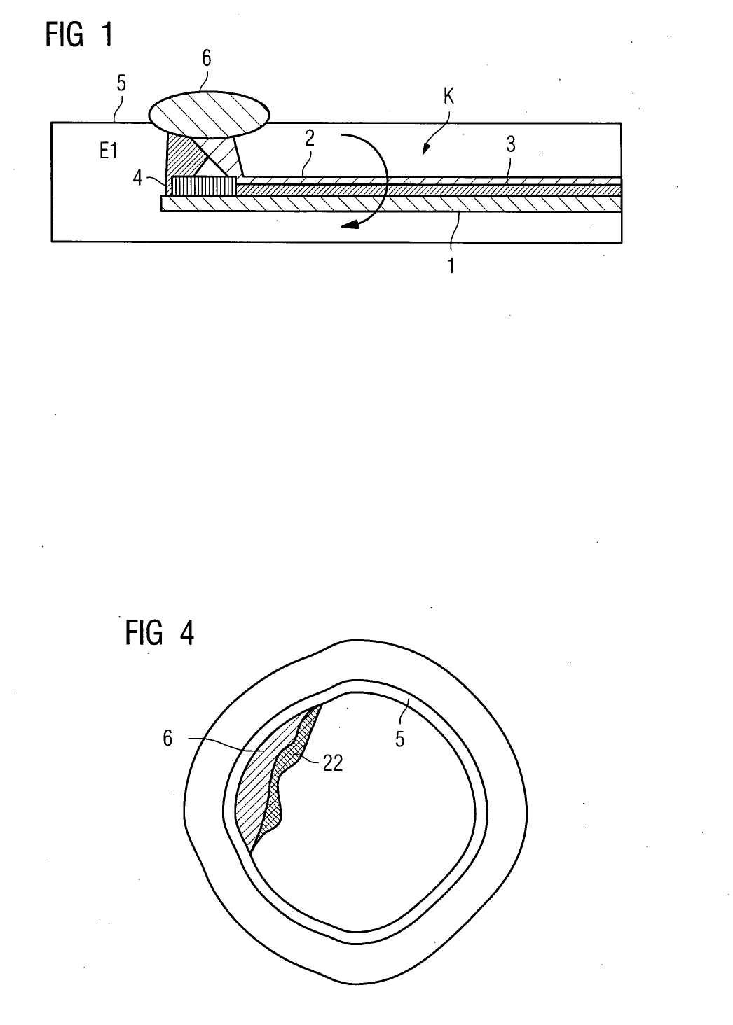

FIG. 1 shows a schematic sectional diagram of a catheter K. In a flexible tube not shown here—preferably grouped into a first group—are rotatably accommodated a first signal line 1, a second signal line 2 and a third signal line 3. Connected to the free end El of the catheter K is the first signal line 1 with a signal detection device 4. The reference symbol 5 identifies a vessel wall on which a deposit 6 marked with a fluorophore is located.

The first signal line 1 and the signal detection device 4 are embodied in the conventional manner so that sectional tissue images are either able to be produced by them using Optical Coherence Tomography, Ultrasound Tomography or Magnetic Resonance Tomography. In the case of Optical Coherence Tomography the first signal line 1 is embodied as an optical fiber at the end of which a mirror is provided as a signal detection device 4. In the case of Ultrasound Tomography an ultrasound transceiver device is advantageously used as a signal detection device 4. In this case the first signal line 1 is an electrical line. In the case of Magnetic Resonance Tomography the signal detection device 4 can for example be an electromagnetic device which is connected to an electrical line as a first signal line 1 with a suitable evaluation device.

The second signal line 2 is always formed from an optical fiber. It is used for detection and forwarding of fluorescence light, which is emitted by a fluorophore accumulated in the deposit 6. With the third signal line 3 for example excitation light for exciting fluorophores on the vessel wall 5 or deposits located there 6 can be beamed in.

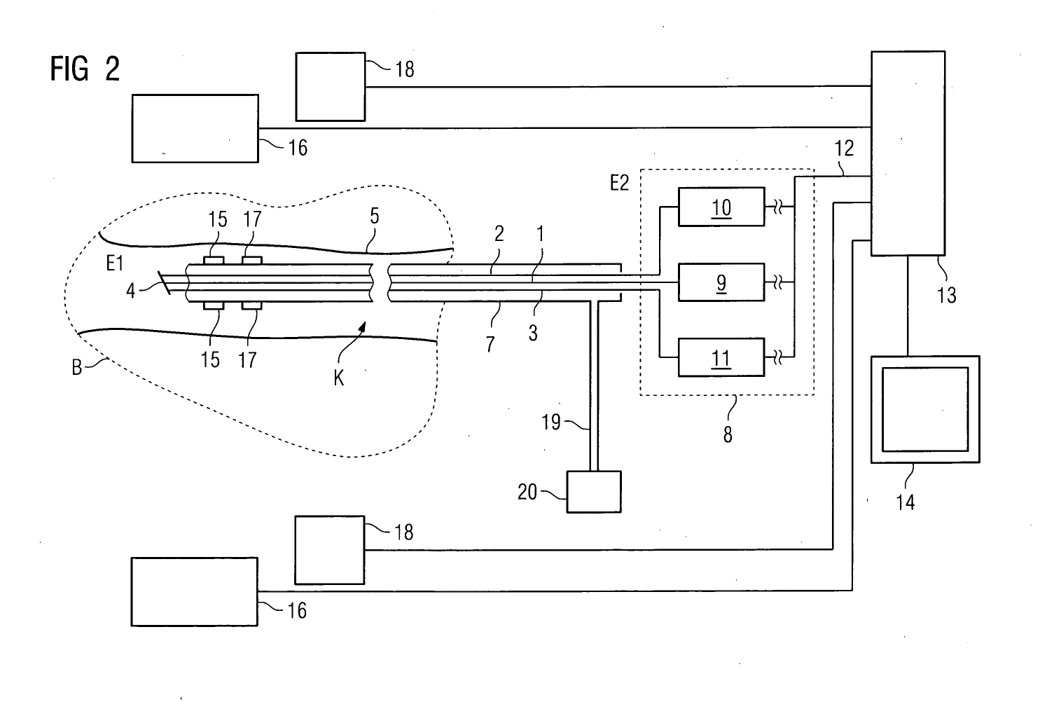

FIG. 2 shows a schematic block diagram of an image generation device. in this case a first signal line 1 embodied as an optical fiber is permanently connected to second 2 and third signal lines 3 also embodied as optical fibers. Provided at the free end E1 of signal lines 1, 2, 3 as a signal detection device 4 is a reflection means for light, e.g. a mirror, with which light can be beamed onto the vessel wall 5 and the light reflected from it can be detected. The line group formed from signal lines 1, 2, 3 is mounted rotatably in a flexible tube 7. Labeled with reference symbol 8 is a rotational drive for rotating the signal lines 1, 2, 3. The first signal line 1 is connected at its other opposite end E2 from the signal detection device 4 to an OCT device 9, with which both light can be coupled into the first signal line 1 and also light reflected from the vessel wall 5 can be detected and is able to be converted into electrical, preferably digitized, signals. Labeled with the reference symbol 10 is a fluorescence light detection device, with which fluorescence light detected via the second signal line 2 is able to be detected. The third signal line 3 is connected to a light generation device 11, with which light of a predetermined wavelength or of a predetermined wavelength range can be beamed onto the vessel wall 5 via the third signal line 3. The signals detected with the OCT device 9 as well as the fluorescence detection device 10 are transferred via a further signal line 12 from a computer 13 to produce an image able to be displayed on a monitor 14. Mounted at the free end E1 of the tube 7 are deflection means 15, which interoperate with deflection devices 16 outside the body, indicated schematically by the reference symbol B. The deflection devices 16 can for example be electromagnets of which the magnetic field strength and direction is able to be controlled in accordance with a predetermined program with the computer 13. The deflection means 15 can for example be embodied as permanent magnets, which, in reaction to the magnetic field formed with the deflection device 16, cause the flexibly embodied free end E1 of the catheter K to bend in a desired direction.

Position sensors are identified by the reference symbol 17. These can involve electromagnetic coils aligned in different directions or similar, which again interoperate with transceivers 18 arranged outside the body B. With the transceivers 18 signals can be beamed onto the position sensors 17 and/or signals emitted by the sensors can be detected. From the detected signals it is once again possible, using conventional algorithms to determine a position of the free end E1 of the catheter K in for example a three-dimensional coordinate system through the arrangement of the transceiver devices 18.

In FIG. 2 the tube 7 forms a line, which is connected via a hose 19 to a fluid feed device 20. In this way contrast media, medicaments and such like can be transported to an opening provided at the free end E1.

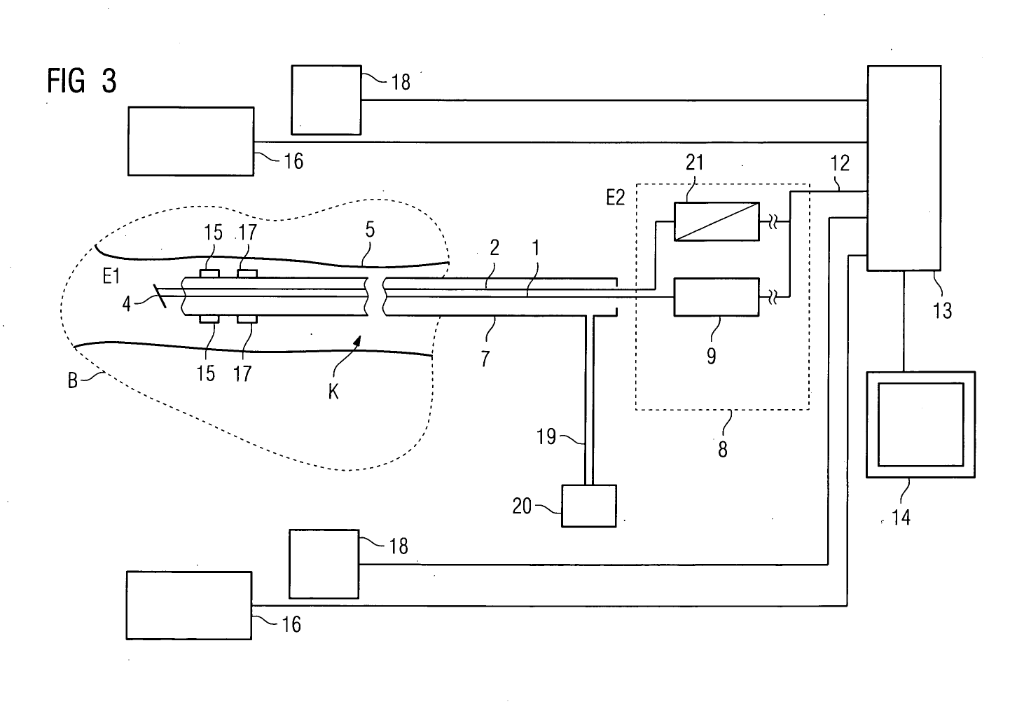

FIG. 3 shows in a further schematic block diagram a second image generation device. In this case the catheter K merely features a first 1 and a second signal line 2 which once again are mounted rotatably in a tube 7. Both the first 1 and also the second signal line 2 are embodied as optical fibers. The first signal line 1 once again connected to the OCT device 9 for generating signals from sectional images of tissue according to the principle of optical computer tomography. In the present exemplary embodiment the second signal line 2 is used both for coupling-in of light of a predetermined wavelength and also for detection of fluorescence light. For this purpose the second fiber is connected at its other opposite end E2 to its free end E1 to a combined light generation and detection device 21. In this way a predetermined pulse of light of a predetermined wavelength or of a predetermined range of wavelengths can be coupled into the second signal line 2 and fluorescence light reflected form the vessel wall 5 are alternately detected.

The function of the image generation devices will now be explained in greater detail with reference to the schematic sectional tissue image depicted in FIG. 4. To produce the image shown in FIG. 4 a sectional image of the tissue is first detected in a conventional manner, for example with the catheter K shown in FIG. 1 using ultrasound tomography. The vessel wall 5 is clearly visible. Using ultrasound tomography, deeper tissue layers lying behind the vessel wall 5 can also be resolved.

To generate an additional fluorescence image 22 the vessel wall 5 is irradiated with light or a predetermined wavelength via the third signal line 3. The predetermined wavelength involves a wavelength suitable for exciting predetermined fluorophores The fluorescence light generated in this way is detected via the second signal line 2 and converted by means of the fluorescent light detection device 10 into electrical, preferably digitized, signals. These signals are then converted in the computer 13 into image information. This image information or the fluorescence image 22 is then overlaid using conventional computational means with the sectional tissue image, so that from the overlaid sectional tissue image thus produced the position and arrangement of the fluorophores and thereby of the pathogenic tissue is able to be detected.

Instead of or simultaneously with the fluorophores, medicaments which are able to be activated with light can also be applied to the pathogenic tissue. For this purpose light with a wavelength or range of wavelengths suitable for activating such medicaments can be beamed onto the vessel wall 5 via the third signal line 3. To this end the light generation device 11 can include means with which light of different predetermined wavelengths is able to be generated. These can for example involve filters, different light sources or similar.

1.-23. (canceled)

24. A medical instrument for introduction into the body of a living being, comprising:
a signal detection device;
an elongated element, comprising:
a first signal line connected to the signal detection device at a free end of the elongated element for detection of first signals for generating a sectional tissue image, and
a second signal line permanently attached to the first signal line, the second signal line embodied as an optical fiber and routed to the free end for transmission of fluorescence light generated inside the body; and
a group formed from the first and the second signal lines mounted rotatably in the elongated element.

25. The medical instrument as claimed in claim 24, further comprising a third signal line embodied as an optical fiber routed in the elongated element to the free end, the third signal line provided for transmission of light of a predetermined wavelength for excitation of fluorophores taken into the body.

26. The medical instrument as claimed in claim 25, wherein the third signal line is permanently attached to the first signal line.

27. The medical instrument as claimed in claim 24, further comprising a third signal line embodied as an optical fiber routed in the elongated element to the free end, the third signal line provided for releasing medicaments provided inside the body.

28. The medical instrument as claimed in claim 27, wherein the third signal line is permanently attached to the first signal line.

29. The medical instrument as claimed in claim 24, wherein the signal detection device is for detection of signals for generating sectional tissue images by means of optical coherence tomography.

30. The medical instrument as claimed in claim 24, wherein the signal detection device is for detection of signals for generating sectional tissue images by means of ultrasound tomography.

31. The medical instrument as claimed in claim 24, wherein the signal detection device is for detection of signals for generating sectional tissue images by means of magnetic resonance tomography.

32. The medical instrument as claimed in claim 24, wherein first signal line being permanently connected to the signal detection device.

33. The medical instrument as claimed in claim 24, further comprising a positioning device at the free end for determining the position in a predetermined three-dimensional coordinate system.

34. The medical instrument as claimed in claim 33, wherein the positioning device comprising a plurality of position sensors.
